(19) United States
(12) Patent Application Publication
(10) Pub. No.: US 2005/0112587 A1
(43) Pub. Date: May 26, 2005

(54) Analyzing biological probes

(76) Inventors: James Vig Sherrill, Oak Ridge, TN (US); Charles Franklin Barnett, Knoxville, TN (US)

Correspondence Address: LUEDEKA, NEELY & GRAHAM, P.C., P O BOX 1871, KNOXVILLE, TN, 37901, US

(21) Appl. No.: 10/721,917

(22) Filed: Nov. 25, 2003

(51) Int. Cl. C12Q 1/68; C12M 1/34

(52) U.S. Cl. 435/6; 435/287.2

(57) ABSTRACT

An analyzer reads biological probes arranged in a probe pattern on a slide carried by a slide carriage. Each probe may or may not generate a probe signal, which is typically fluorescent light, to indicate prior exposure to a predetermined substance or absence of such prior exposure. A detector includes sensors arranged in a first pattern. The probe pattern is denser than the first pattern and includes plurality of first patterns. The detector can sense multiple subsets of the probes within a probe pattern. The probes are contained in wells configured to facilitate reading of the probes, and alignment indicia may be used to check alignment of the slide with the detector.

### FIELD OF THE INVENTION

The present invention relates to the field of analyzing biological probes and particularly relates to an apparatus and method for analyzing multiple subsets of probes emitting light, for example fluorescent and bioluminescent probes.

### BACKGROUND OF THE INVENTION

Biological probes have been designed to identify a particular biological substance by attaching to that substance. In use, the probes are attached to a substrate, and an unknown material is treated with markers or tags before applying the unknown material to the probes. Often, a fluorescent tag is attached to the unknown material, and the material is exposed to the probes. If the unknown material includes a particular type of bacteria, and if a probe for those bacteria is present, the bacteria will attach to the probe. Then, the tag carried by the bacteria may be detected. Examples of detectors for biological probes are described in the U.S. Pat. Nos. 6,197,503 and 6,448,064. Another technique attaches biological probes that include fluorescent material that will emit light only if the probe is exposed to its particular target material.

Expense is a primary stumbling block for applying this technology to a broad base of industrial and commercial applications. Often, highly skilled technicians are required to prepare the slide, appropriately expose it to a material in question, and read the slide. Typically, the slides are expensive and the machines used to read the slides are even more expensive. Thus a need exists for an inexpensive fast mechanism and method for reading biological slides by trained personnel who are not necessarily highly skilled in the biological field.

### SUMMARY

The present invention provides an apparatus and method for economically producing, using and reading biological slides, meaning slides or other support structure carrying biological probes. The technology of the present invention will lower the cost of using biological slides to the point that everyday commercial applications will be possible. For example, restaurants and groceries may routinely check their meat and other foods for specific types of bacteria.

In accordance with present invention, an analyzer is provided for reading biological probes. The analyzer includes a housing, and a slide carriage is mounted for movement within the housing. A slide is positioned on the slide carriage, and a plurality of probes are disposed on the slide in a defined pattern. Each probe may generate an electromagnetic probe signal, to indicate prior exposure to a predetermined substance. Alternatively, the probe signal may not be produced, which indicates the absence of prior exposure to a predetermined substance. The detector is configured to detect probes signals from a plurality of probes located in a defined pattern on the slide. Also, the defined pattern is denser than the first pattern of the detector and is configured in a shape corresponding to a plurality of first patterns so that the detector can sense multiple subsets of the probes within a probe pattern on the slide.

In a preferred embodiment the detector includes a plurality of sensors arranged in the first pattern, and each sensor is disposed for detecting a probe signal from a single probe set. The plurality of sensors on the detector is aligned with a subset of the probes on the slide when the slide is being read. Preferably the sensors are arranged on the detector in a first pattern and the probe pattern on the slide is arranged in the form of interlaced first patterns. Thus, the detector may be aligned with a first pattern of sensors aligned with a first pattern of probes constituting a subset of the probe pattern. All of the probes on the slide may be read by sequentially moving the detector or the slide or both, one relative to the other, from one subset to another subset of probes on the slide. In one embodiment the first pattern and the probe pattern both comprise a plurality of locations arranged in rows and columns. The number of rows and columns in the probe pattern is a multiple of the number of rows and columns in the first pattern. For example, if the first pattern has 5 rows, the probe pattern may have 10 rows or 15 rows, etc.

In another embodiment the first pattern is in the shape of a pie section and the probe pattern is arranged on the slide in a radial configuration about a center point. Again, the probe pattern preferably is denser than the first pattern, and most preferably the probe pattern includes multiples of the first pattern. With the probe pattern arranged about a center point on the slide, the slide may be read by aligning the detector with one first pattern on the slide, reading the probes with which the detector is aligned, relatively moving the slide and the detector to align the detector with subsequent sets of probes on the slide arranged in the first pattern, reading the second set of probes, and continuing to relatively move the detector and slide and read additional probes, preferably until all probes on the slide are read.

In accordance with another aspect of the invention, the analyzer includes a source of electromagnetic radiation (preferably a scanning laser) for illuminating the probes at selected times and the source is extinguished at other times so that the probes are either illuminated or not. The probes are preferably constructed in part from a fluorescent material to indicate exposure to a selected material. Thus, certain probes emit a probe signal in the form of fluorescent light after being illuminated by the source and other probes do not. In one embodiment, the probes that include a fluorescent material were made by first placing a material at the probe location that will bind to another specific material, such as nucleotides or DNA. Then, an unknown material has fluorescent markers attached to it, and then both the unknown material and the fluorescent markers or tags are exposed to the probes. If the unknown material is the material to which the probe binds, then the unknown material will bind to the probe and the fluorescent marker will be present on the probe on the slide. If the unknown material does not bind to a particular probe, then that particular probe will not include fluorescent material. Therefore, the presence or absence of fluorescent material on each probe indicates the presence or absence of particular types of material, such as a particular strain of bacteria, in the unknown material.

In an alternate embodiment, all of the probes have a fluorescent material attached to the probe, but the fluorescent material will not fluoresce unless and until the probe is exposed to the particular substance that a particular probe is designed to detect. Thus, if such particular probe fluoresces when exposed to the source, it means the probe was exposed to a particular substance. If such probe does not fluoresce when exposed to the electromagnetic radiation of the source, it means that the probe has not been exposed to the particular substance, and the fluorescent material will not fluoresce even though it resides on the probe and has been illuminated.

In order to align the detector with the slide, alignment indicia is preferably disposed on the slides to enable proper alignment. Preferably one or more indicia are placed on the slide in the location of one or more probes. The indicium produces a probe signal in the form of light that is detected by the sensors. The detector aligns itself by aligning the detector sensors over the indicia in the probe locations. Preferably the drive mechanism for the slide carriage includes an X, Y, Z, and R drive mechanism for moving of the slide carriage in X, Y and Z directions, which are non-parallel directions, preferably orthogonal directions. Also, the drive mechanism rotates the slide carriage about an axis, “R”. Using the drive mechanism, the detector is moved until the expected light from the alignment indicia is received by the expected sensors. Since the alignment indicia is controlled to contain a specific amount of light producing material that produces a specific amount of light, and since the exact position of the alignment indicia is known, it is also known that the alignment indicia will produce a specific reading (the detection signal) in specific sensors when the detector is properly aligned over the slide.

Thus, by moving the detector with respect to the slide until the expected specific amount of light is received in the known specific sensors, the slide is aligned with the detector. (As used herein, mechanism and mechanisms are synonymous, and either may have many parts or just one part.)

### BRIEF DESCRIPTION OF THE DRAWINGS

Additional aspects and advantages of the present invention may be understood by reference to the following Detailed Description when considered in conjunction with the attached drawings in which:

FIG. 1 is a perspective outside the view of the biological slide analyzer;

FIG. 2 is a somewhat schematic side view of a cassette and a slide carriage extending through an opening in the housing;

FIG. 3 is a schematic cutaway view of the cassette showing the slide carriage and the drive mechanism for the slide carriage;

FIG. 4 is a block diagram illustrating the position and relationship between the detector and the slide;

FIG. 5 is a schematic diagram of a top view of a detector in which the individual sensors are shown as if the detector was transparent;

FIG. 6 is a top view of a slide showing multiple probe locations on the slide;

FIG. 7 is a top view of another detector, again shown as if it were transparent so that the sensors are shown;

FIG. 8 is a top view of another slide with the probe locations corresponding to the pattern of the sensors in FIG. 7;

FIG. 9 is a top view of another detector, again shown as if it were transparent so that the sensors are shown;

FIG. 10 is a top view of a circular slide with the probe patterns concentrically arranged about the center of the slide and corresponding to the pattern of the detectors in FIG. 9;

FIG. 11 is a broken away schematic side cross sectional view of a well constructed in a transparent slide for containing a probe;

FIG. 12 is a broken away schematic side cross sectional view of another well constructed in a reflective slide for containing a probe;

FIG. 13 is a broken away schematic side cross sectional view of another well in a slide constructed of a material that absorbs the illuminating light; and

FIG. 14 is a block diagram showing a data processor connected to the various components of the analyzer.

### DETAILED DESCRIPTION OF THE DRAWINGS

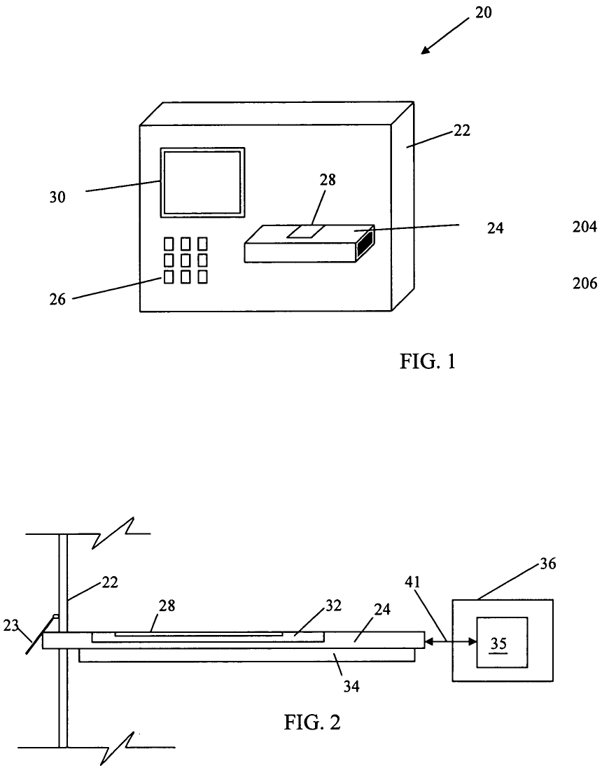

Referring now to the drawings in which like reference characters designate like or corresponding parts throughout the several views, there is shown in FIG. 1 a biological slide analyzer 20, including a housing 22 that contains the biological slide analyzer 20 and protects the analyzer 20 from outside ambient light. A cassette 24 is mounted on the front surface of the housing 22 and moves into and out of the housing 22 carrying a biological slide 28. The operation of the analyzer 20 is controlled by a user through the keypad 26 and a display 30. The user enters commands and inquiries through the keypad 26 and receives information from the analyzer 20 through the display 30. The biological slide 28 is carried into the analyzer 20 by the cassette 24 in response to commands from the user through keypad 26. The biological slide 28 is typically a DNA micro-array. Typically, oligonucleotides are disposed on the biological slide 28 in a pattern or array. The oligonucleotides that are disposed on the slide 28 (or any other reactive agent that is placed on the biological slide 28) are referred to herein as probes.

The slide 28 has been prepared in advance before it is placed into the analyzer 20. For example, a slide may be prepared with a plurality of the probes formed on the slide with each probe designed to bind with a specific type of biological material. In one embodiment, the biological probes are constructed using a detector material with a fluorescing material associated with the detector material. Each probe may have different detector material that reacts or detects different substances. The fluorescing material is associated with the detector material such that the fluorescing material transmits fluorescent light from the probe only when the detector material has contacted the particular substance it is designed to detect. Thus, if the particular substance is present, the probe will emit fluorescent light when illuminated by an appropriate source of electromagnetic illumination. For example, the probe may be designed to fluoresce when a particular food bacteria is exposed to the probe.

In an alternate embodiment, the probes are designed to detect and attach to particular substances, but the fluorescing material is attached to the target bacteria, not the probe. A material of interest, such as a food product, is then treated with fluorescent markers that attach to the food product and extraneous substances that may be present in the food products. For example, if bacteria are present in the food product, the fluorescent markers attach to the bacteria. The treated material of interest is then exposed to the slide 28. If one of the probes is designed to bind to salmonella bacteria, and if salmonella is present in the food product, the salmonella bacteria will bind to that particular probe of the slide 28. The salmonella bacteria will carry the fluorescent markers and, thus, the presence of the salmonella on the probe may be detected by detecting fluorescent light from the fluorescent markers on the salmonella, which is bound to the probe. This one example illustrates the many types of slides of that may be prepared for the analyzer 20. A common feature of each slide is that it includes a plurality of spots that will produce signals, such as light, and thereby provide information.

Referring to FIG. 2, a schematic side view of the cassette 24 is shown. In this view, the housing is shown broken away and the cassette is represented in a block diagram form. The cassette 24 includes a slide carriage 32 that holds and moves the biological slide 28 as needed by the analyzer 20. A cassette mount 34 holds the cassette 24 and allows it to slide into and out of the housing 22. When the cassette 24 slides completely into the housing 22, a hinged door 23 (optional) closes to prevent any outside ambient light from entering the housing 22 and interfering with the operation of reading the biological slide 28. If a door is not used, the cassette 24 is dimensional and configured to block ambient light from entering the housing 22 when the cassette 24 is closed. A cassette drive mechanism 36 moves the cassette 24 into and out of the housing 22, and preferably includes a motor 35 and a mechanical drive train 41, such as a screw drive train or belt drive mechanism.

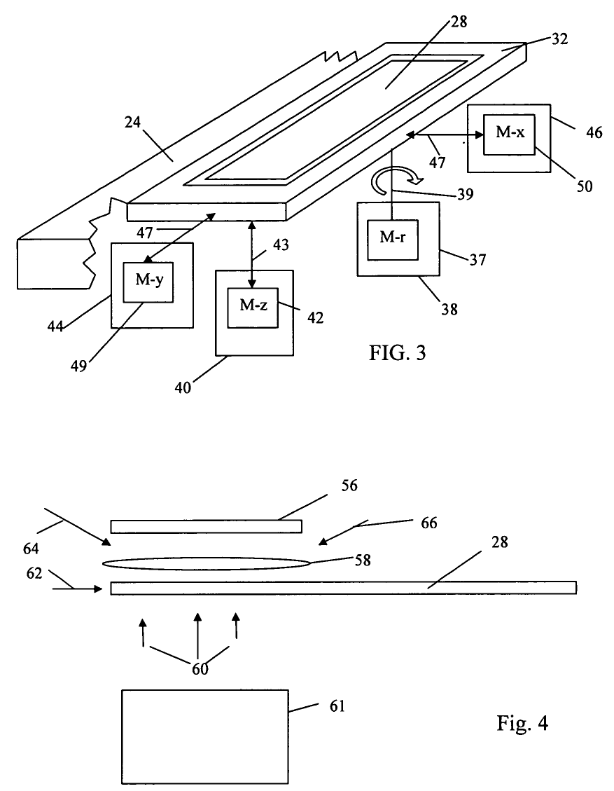

A more detailed view of the slide carriage 32 is shown schematically in FIG. 3, and the cassette 24 is shown broken away to reveal the carriage 32. One purpose of the carriage 32 is to move the slide 28 into proper position for being read or examined. The carriage 32 is mounted on a rotational drive mechanism 37 that includes a motor 38 and a rotational drive train 39, such as a drive shaft, chain or belt.

The rotational drive mechanism 37 is configured to rotate the carriage 32 through 360 degrees of rotation in either direction as indicated by drive train 41. The rotational drive mechanism 37 includes an appropriate motive mechanism such as drive motor 38. Operating above or on top of the rotational drive mechanism 37 is a vertical drive mechanism 40 that drives the carriage up and down in the vertical direction, the Z direction. Again, the vertical drive mechanism 40 includes a motor 42 and a drive train 43, such as a drive screw, chain or belt. Drive motor 42 provides force to move the mechanism 40 and thereby move the carriage 32 up and down. On top of the vertical drive mechanism 40, a horizontal Y drive mechanism 44 is mounted and includes a drive motor 49 and a drive train 47. The horizontal Y drive mechanism 44 moves the carriage 32 horizontally in the Y direction. Finally, a horizontal X drive mechanism 48 is mounted above the horizontal Y drive mechanism 44. The drive mechanism 48 moves the carriage 32 in the X direction as indicated in FIG. 3 and is likewise provided with a drive motor 50 and the drive train 47.

While it is preferred to provide the movement of the slide 28 in the manner previously described, in alternate embodiments, different types of movement may be provided. For example, in some applications, it may be desirable to provide three-dimensional movements along non-perpendicular directions. Also, a different order of movement might be desirable in other embodiments. In the above example, rotational movement caused by drive mechanism 37 is the base form of movement upon which all other movements are built. In alternate embodiments, the rotational movement could be provided as the highest order of movement. That is, the rotational drive mechanism 37 would be mounted on top of the other portion of the drive mechanism. In the embodiment shown in FIG. 3, the X drive mechanism 46 is now the highest order drive mechanism. Thus, if the rotational drive mechanism 37 is moved and is placed on top of the X drive mechanism 46, the rotational drive mechanism 37 will become the highest order drive mechanism.

Also, the movement of the slide 28 is relative to the detector 56 (FIG. 4), which can be produced by moving the slide 28 or the detector 56 or both. When describing the motion of the slide herein, such motion could be obtained by moving either the slide or the detector.

Referring now to FIG. 4, the process of reading a slide 28 is illustrated. FIG. 4 is a schematic side view of a slide 28, a detector 56 and conditioning optics 57. As previously mentioned, probes are placed on the slide 28 and are illuminated by light, such as light ray 63 produced by scanning laser 61, that illuminate the underside of slide 28, light ray 62 that illuminates the side of the slide 28 and light rays 65 that illuminate the upper side of the slide 28. In this illustration the laser 61 represents a laser, conditioning optics and mechanical drives that filter, collimate and scan the laser beam. It is preferred that the light be provided by a scanning laser 61, but non-coherent light forms will perform the desired function as well. The light rays 63, 62 and 65 excite the probes on the slide 28 and, in some cases as described below, the presence of probes will be indicated by fluorescent light produced by the probes. Typically, the external light source (laser 61) is turned off and the detector 56 is allowed to detect fluorescent light immediately after the external light rays 63, 62 and 65 are extinguished. However, alternately, the fluorescent light may be detected during exposure to the external light source.

The conditioning optics 57 may include lenses and filters. Preferably the conditioning optics 57 will filter ambient light and light at the frequency of the light applied to the slide 28, such as the frequency of light rays 63, 62 and 65. Thus, conditioning optics 57 is designed to filter out the light rays 63, 62 and 67 preventing it from reaching the detector 56.

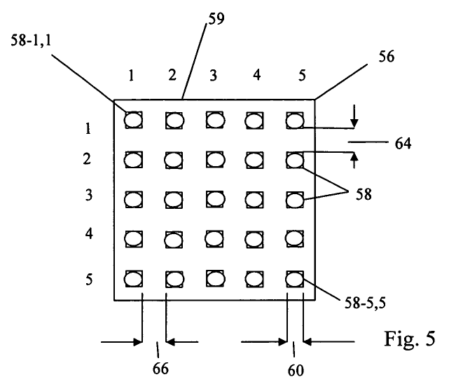

A view of the top side of detector 56 is schematically shown FIG. 5 as if the detector 56 was transparent. In this embodiment, the sensors 58 are actually on the bottom side of the detector 56, but to illustrate alignment with the slide 28, it is preferred to illustrate the detector 56 with a top view showing the sensors 58 as if one could see through the detector 56. However, the sensors 58 may be on any surface of detector 56.

The detector 56 in this embodiment includes an array of twenty-five sensors 58 arranged in five rows and five columns. The row and column numbers are labeled along the left and top side of the detector 56, respectively. For reference purposes the top left sensor 58 is positioned at a row 1, column 1 and therefore has a position of 1, 1. For convenient reference purposes, this sensor will be labeled sensor 58-1,1. The sensors 58 are formed on a substrate 59 that is preferably constructed of a semiconductor material, such as silicon. The sensors 58 are each light sensitive photo diodes that a produce an electrical signal in response to receiving light. In this particular embodiment, the sensors 58 are preferably round having a diameter 60 of about 1.0 mm. The sensors in this embodiment are separated in the Y direction by a Y distance 64 equaling about 0.5 mm and are separated in the X direction by an X distance 66 equaling 0.5 mm. These precise dimensions are given for purposes of illustration only are not intended to limit the scope of the invention. Also, the detectors 58 may be rectangular, oval, or other shapes.

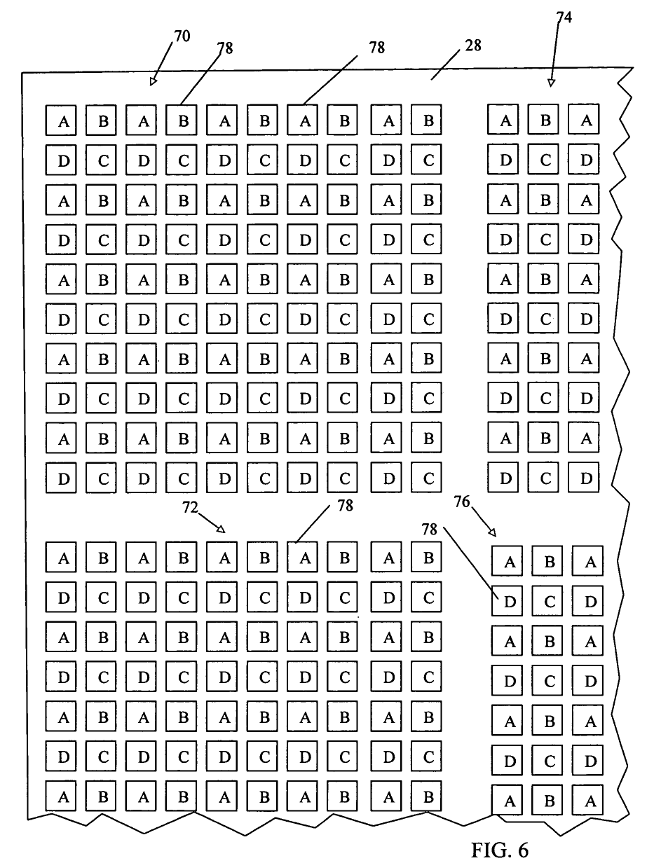

Referring to FIG. 6, a more detailed view of slide 28 is shown. For clarity of illustration, FIG. 6 is not drawn to scale. The slide 28 shown in FIG. 6 includes a plurality of arrays 70, 72, 74 and 76 of probes 78. Each of the arrays 70-76 of probes 78 has a density of four times the density of the array of sensors 58 in the detector 56. The arrays 70, 72, 74 and 76 include probes 78 with each array arranged in 10 columns and 10 rows of probes 78. Referring to the upper left corner of array 70, the sensors 78 are arranged in groups of four sensors labeled A, B, C, and D. This grouping repeats in both the X direction and the Y direction. Several of the sensors and 78 have been labeled to show the repeating pattern of the four sensors A, B, C, D.

To read the array 70, the slide 28 is moved until the detector 56 is positioned over the array 70 with sensor 58-1, 1 positioned over the probe labeled “A”. The array of sensors 58 is aligned with the probes 78 such that each sensor 58 will read an “A” probe 78 when the sensor 58-1, 1 is positioned directly over the sensor 78 labeled “A” in the upper left-hand corner of array 70. After all of the “A” probes 78 are read, the slide 28 moves to the left and, in a relative sense, the detector 56 moves to the right and positions sensor 58-1, 1 over the “B” probes 78 in the upper left-hand corner of array 70. Likewise, all other sensors 58 are positioned over “B” probes 78 in the array 70. After the “B” probes are read, the slide 28 moves upwardly, and in a relative sense, the detector 56 moves downwardly in the Y direction and positions the sensor 58-1, 1 over the “C” probe 78 in the upper left-hand corner of the array 70. Again, all of the other sensors 58 are positioned over the other “C” probes 78 in the array 70 and the “C” probes are read. Finally, the slide 28 moves right and, in a relative sense, the array of sensors 58 are moved to the left positioning sensor 58-1, 1 over the “D” probes in the upper left-hand corner of the array 70. The other sensors 58 are positioned over the other “D” probes 78 and the “D” probes are read. In this embodiment, only the slide 28 moves, but in other embodiments, the detector 56 may be constructed to move, or both the detector 56 and slide 28 may move.

After array 70 and has been completely read, the detector 56 is positioned over the array 72 and the reading process is repeated. Thereafter, arrays 74 and 76 are read in the same manner. In FIG. 6, the slide 28 has been broken away, but it will be appreciated that the pattern of arrays may be continued down the slide 28.

Referring again to FIG. 3, the relative motion between the slide 28 and the detector 56 may be understood. In this embodiment the X drive mechanism 48 and the Y drive mechanism 44 are the primary mechanisms used to move the slide 28 relative to the detector 56 and properly positioned in the sensors 58 over the probes 78. However, the rotational position of the slide 28 relative to the detector 56 is also important. If the slide 28 is slightly misaligned rotationally with respect to the detector 56, the slide 28 cannot be properly read. Likewise, the distance between the slide 28 and the detector 56 is important to achieve repeatable quality results. Thus, the slide 28 may be moved in the Z direction using the Z drive mechanism 40 to adjust the distance between the detector 56 and the slide 28.

The positions of the probes 78 on the slide 28 are very precise and the positions of the sensors 58 on the detector 56 are very precise, but the relative positioning between the slide 28 and the detector 56 is less precise because the slide 28 must be removed and moved during operation of the analyzer 20. In practice, once the analyzer 20 is calibrated properly, the detector 56 and the slide 28 may be accurately and properly positioned without alignment checks. However, in the preferred embodiment, alignment checks are provided. Preferably on every slide, the first and the last “A” probes 78 are seeded with material that will fluoresce brightly. For example, referring to array 70 in FIG. 6, the “A” probes 78 in the upper left-hand corner and the lower right-hand corner are seeded with fluorescing material. Preferably, this material will fluoresce more brightly than a typical fluorescing probe. When the slide 28 is first positioned under the detector 56, the light from the two “A” probes is detected and the slide 28 is rotated and moved vertically to place the slide in the optimum position. Preferably, the slide 28 is first rotated and moved in the X and Y direction to maximize the light that is received by sensors 58-1, 1 and sensor 58-5, 5. Then, the slide is moved in the vertical or Z direction until the slide 28 is positioned at a proper distance from the detector 56. In some embodiments, the conditioning optics 57 (FIG. 4) will focus the light from the probes 78 onto the sensors 58. In that type of the instrument, the proper distance between the slide 28 and the detector 56 is a distance that is required to place the sensors 58 in the proper focal plane. In other embodiments, the conditioning optics 57 does not include lenses or other focusing devices and the intensity of light received by the sensors and 58 is inversely proportional to the distance between the detector 56 and the slide 28. In such case, the slide 28 is moved in the vertical direction until a predetermined light intensity is detected by the sensors 58-1, 1 and 58-5, 5. A known amount of fluorescing material has been positioned in the first and last “A” sensors of array 78, and the amount of fluorescent light produced by those two probes is carefully controlled so that a predetermined light intensity level corresponds to a desired distance between the slide 28 and the detector 56. Thus, once in the slide 28 is properly positioned in the X, Y, and R are positions, the slide 28 is moved up and down in the Z direction until the detectors 58-1, 1 and 58-5, 5 sense the aforementioned predetermined light intensity level corresponding to the desired distance. Thus, the slide 28 is properly positioned in the Z direction.

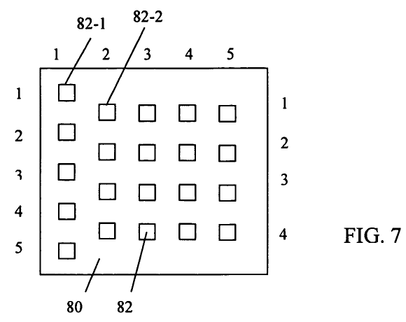

Referring now to FIG. 7, an alternate detector 80 is shown. Again, the detector 80 is shown from the top side and the sensors 82 are illustrated as if one could see through the detector 80. It will be understood that the sensors 82 are actually disposed on the underside of the detector 80. However, the sensors 82 could be disposed on any side or surface of the detector 80. In detector 80, a plurality of sensors 82 are arranged in rows and columns. However, in the detector 80, the pattern is irregular. To illustrate irregularities, the first column includes five sensors 82 and the remaining columns include 4 sensors 82. Numbers 1-5 across the top of the detector 80 label the columns of sensors 82. Numbers 1-5 along the left side of the detector 80 label the rows of the first column and the numbers along the right side of FIG. 7 label the four rows of the four columns 2-5. Columns 2-5 are shifted downwardly in the Y direction to center the rows of columns 2-5 with the spaces between the sensors 82 in column one. Thus the rows of columns 2-5 are misaligned with the sensors 82 in column one. In this embodiment, the different numbers of sensors 82 in a column and the misalignment of the rows represent irregularities.

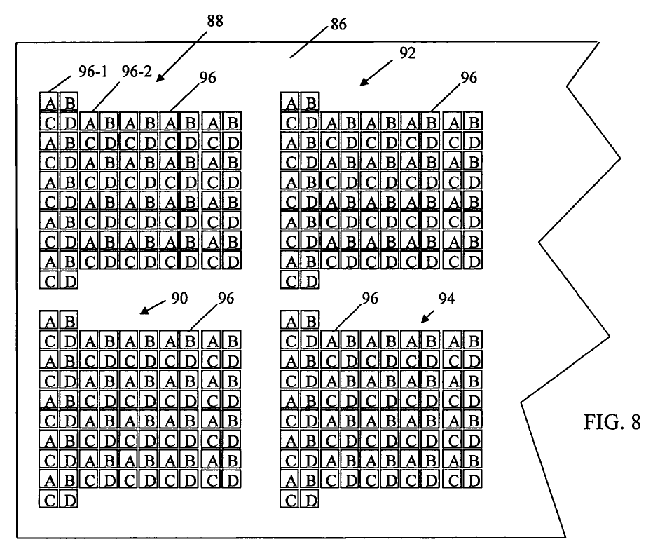

The irregular pattern of sensors 82 and the detector 80 are configured to read the irregular pattern of probes 96 disposed on the slide 86 shown in FIG. 8. The probes 96 are arranged into four arrays 88, 90, 92, 94. To read the probes 96, the slide 86 is positioned to align probe 96-1 with sensor 82-1. Also, probe 96-2 is aligned with sensor 82-2. Then, the slide 86 is shifted first right, then down, then left to read the probes B, C, and D in the manner previously described in reference to FIG. 6. The irregularly shaped pattern of sensors 82 and probes 96 assists in the original alignment of the probes 96 and sensors 82, particularly, during calibration.

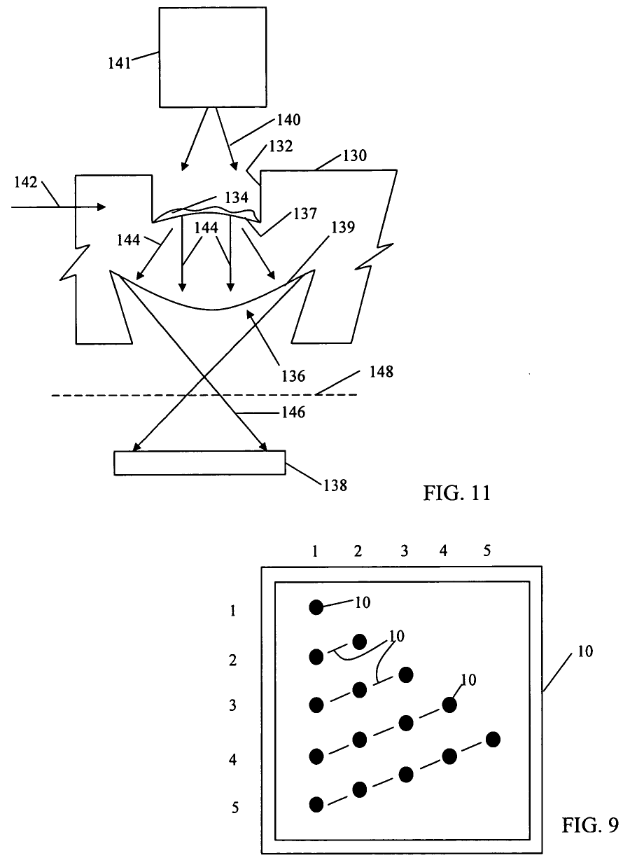

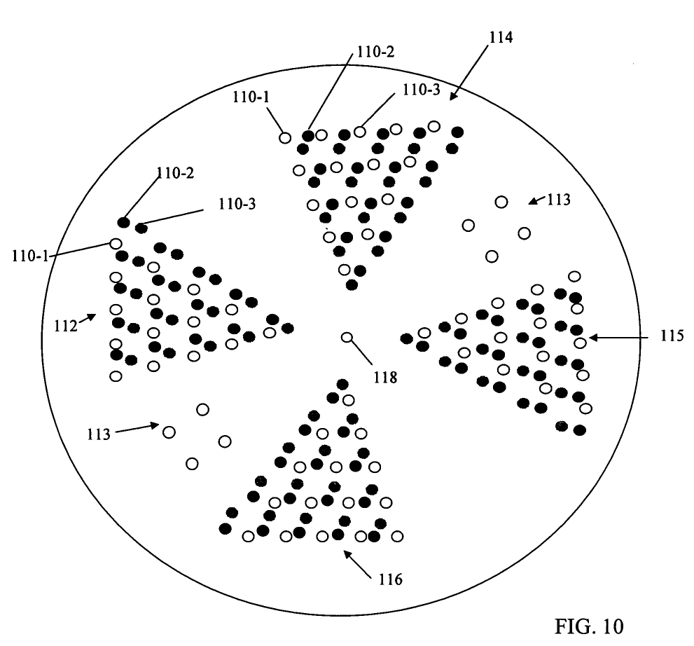

Referring to FIGS. 9 and 10, a detector 100 and a slide 102 are shown representing another embodiment. The slide 102 is circular or disk shaped, and referring to FIG. 9, the sensors 104 are arranged in five columns and five rows. The numbers across the top of FIG. 9 label the columns and the numbers on the left side of FIG. 9 label the rows. In this embodiment, the pattern is irregular in that each column and each row includes a different number of sensors. In addition, the rows are not horizontal. Instead, they are inclined as indicated by lines 106 which have been added for illustration purposes only to ensure that the rows 1-5 are properly identified.

Referring again to FIG. 10, probes 110 are arranged in patterns corresponding to the pattern of sensors 104. The probes of 110 are arranged in four arrays 112, 114, 115, and 116. Each of these four arrays includes three patterns of probes 110 corresponding to the single pattern of detectors 104. Referring to the left side of FIG. 10, the probes 110-1 (illustrated as white dots) represent a first pattern corresponding to the pattern of detectors 104 shown in FIG. 9. The probes 110-2 (illustrated as black dots) represent a second pattern and the probes 110-3 (illustrated as gray dots) represent a third pattern, where both the second and third patterns correspond to the pattern of sensors 104 of FIG. 9.

The patterns in each of the arrays 112-116 are oriented in a radial direction with respect to the center 118 of the circular slide 102. A radial configuration does not require that any row of probes 110 be radial with respect to the center 118; it only requires that the patterns be oriented on the slide 102 such that the patterns of probes may be aligned with the pattern on the detector(s) by rotation about the center 118, plus linear X-Y movement. That is, all of the patterns substantially point to the center 118 of the slide 102. Thus, the slide 102 may be read by a combination of rotating the slide 102 and shifting the slide 102 in the X and/or Y direction. To read the slide 102, the detector 100 is first positioned over the array 112 with the sensors 104 positioned over the probes 110-1 (illustrated by the white dots). After the probes 110-1 are read, the slide 102 is rotated counterclockwise until the detectors 104 align with the probes 110-2 (illustrated by black dots). After the probes 110-2 are read, the slide 102 must be rotated slightly in a clockwise direction and the slide must be shifted to the left until the probes 110-3 are aligned with the detectors 110. When shifting between subsets of probes 110, the slide may also be moved in a linear direction, such as, in the X and Y direction with no rotation. That is, the slide 102 could be moved to the left and up to align the sensors 104 with the probes 110. Of course, the patterns 110 must be positioned to allow such movement, and in such case at least one pattern will not be oriented radially with respect to center point 118. This linear movement may be accomplished using the X and Y drive mechanisms 48 and 44, respectively.

After the array 112 has been read, the slide 12 is rotated in a counterclockwise direction and it is shifted in the X and Y directions until the probes 110-1 (the white dots) of array 114 are aligned with the sensors 104 of the detector 100. Then, the slide 102 is rotated or shifted in the X, Y direction, or both, to read all three patterns in the array 114 in the same manner as previously described. In like manner, arrays 115 and 116 are read.

The arrays and patterns illustrated in FIG. 10 were deliberately chosen to illustrate extreme arrangements of the probes that are possible with the analyzer 20 of the present invention. In a simplified embodiment, slide 102 is arranged with symmetrical patterns of probes 110 (such as arrays 113 and 115) such that the probes are read by simply rotating the circular slide 102 about its center 118. This simplified pattern would have the advantage of easier alignments and easier movements, but it would sacrifice density. By providing patterns of probes on a circular slide that are irregular, the density of the probes may be increased, but the complexity of moving from pattern to pattern on the slide is increased and will typically require both rotational movements and movements in the X and Y directions. The complexities created by irregular and dense patterns are difficult to visualize but are relatively easy to control using data processors and motion control techniques. Thus, the disadvantages of a complex pattern on a circular slide are offset by the advantage of increased density. Depending upon the application, irregular, dense, complex patterns may be preferable.

In the illustration of FIG. 10, large gaps were left between the arrays 112-116. These gaps are intended to aid illustration. In practice, one typically would not leave so much of the slide 102 unoccupied. Instead, the irregular patterns would run together and maximize the density of the probes 110 on the slide 102.

Referring now to FIG. 11, a detailed side of view of the slide 130 is shown. In this view, the slide 130 has been cut away and shows only a single well 132 containing a single probe 134. The slide 130 actually includes many wells and probes are arranged in a pattern as previously discussed. Beneath the well 132 and the probe 134 is a lens or lenses 136 that are designed to collect and focus light on a detector 138. Preferably, the lenses 136 includes an upper convex surface 137 and a lower convex surface 139 that together form the convex lens 136. In operation, the probe 134 is first illuminated by light from the top, such as light rays 140 produced by a scanning the laser 141, and the probes 134 may also be illuminated by a side light passing through the slide 130, as illustrated by light ray 142.

After the probe 132 has been illuminated, the external light sources are extinguished and fluorescent light is produced by the probe 134 as illustrated by light rays 144. The lens 136 may be greater in diameter than the well 132 and extend partially around the probe 134 so that the lens 136 collects light emanating from the probe in multiple directions and directs the light to the detector 138 as indicated by the light rays 146.

Preferably, the detector 138 is positioned in the image plane of the lens 136 to maximize the energy that is collected and concentrated on a detector 138. In addition, conditioning optics 148 may be provided between the detector 138 and the probe 134. The conditioning optics 148 preferably includes a filter that blocks light having a frequency ambient light or the illumination light as represented by light rays 140, 142. The filter of the conditioning optics 148 will transmit light having a frequency equal to that of the fluorescent light emanating from the probe 134 as illustrated by light rays 144. Optics 148 preferably also include a lens or lenses that directs light to the detector 138, such as by producing an image of the probe 134 on the detector 138.

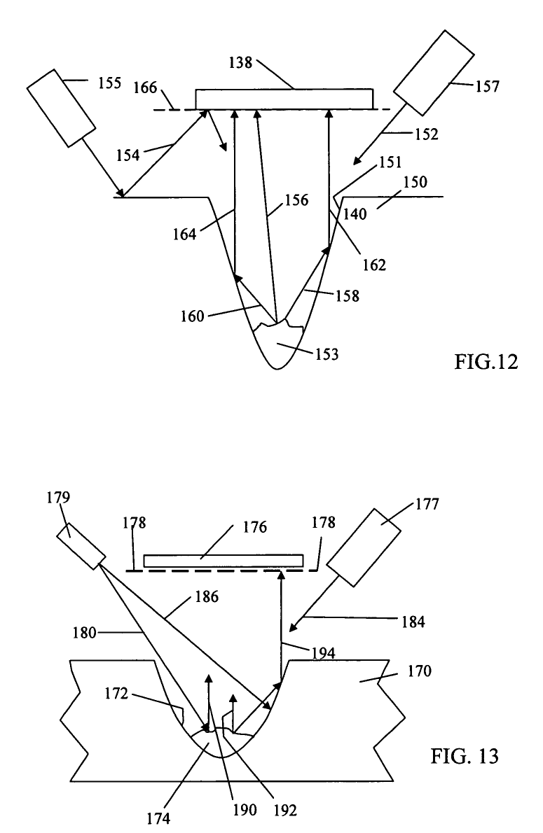

Referring now to FIG. 12, biological slide 150 represents an alternate embodiment of a slide for use in the analyzer 20. The slide 150 is shown in schematic cross section broken away to illustrate a single well 151. A probe 153 is disposed at the bottom of the well 151, and as in prior embodiments, the probe 153 is constituted to emit fluorescent light if it has been exposed to its target, typically a DNA strand. To stimulate the probe 153, it is exposed to excitation light which is illustrated in FIG. 12 by light rays 152, that are preferably produced by a scanning laser 157, and light rays 154, that are preferably produced by a scanning laser 155. In this embodiment, the well 151 is highly reflective, preferably substantially totally reflective of both the illumination light and fluorescent light. Therefore, light entering the well from above will be reflected downwardly until it excites the probes 153 As indicated by light rays 154, some of the light is reflected from the detector 138 will enter the well 151 and strike the probe 153, and such light may be reflected one or more times by the well 151 until it strikes the probe 153.

After exposing the probe 153 to illuminating light such as light rays 152 and 154, fluorescent light is produced by the probe 153 as represented by light rays 156, 158 and 160. Some of the fluorescent light will project directly toward the detector 138 and strike the face of the detector 138, penetrating the detector 138. Fluorescent light as illustrated by light rays 158 and 160 will be transmitted by the probe 153 in the direction of the walls of the well 151. Upon striking the well 151, the light will be redirected toward the detector 138 as indicated by the light rays 162 and 164 representing light rays reflected from the well 151. The well 151 has a parabolic shape, or a modified parabolic shape designed to maximize the amount of fluorescent light that is reflected toward the detector 138. In practice, the parabolic shape of the well 151 is dictated by the position of the top surface of the probe 153. The parabolic shape of the well 151 is configured to maximize the amount of light that is reflected onto the detector 138, where the light is transmitted from the top surface of the probe 153 in substantially all upward directions.

Conditioning optics 166 are also provided to further condition of the fluorescent light emitted by the probe 153 and the illumination light represented by light rays 152 and 154. For example, in a preferred embodiment, the conditioning optics 166 would include a filter designed to totally block light having a frequency of the illumination light rays 154 and 152, but the filter would totally pass light having a frequency of the fluorescent light emitted by the probe 153, such as light rays 156, 158 and 164. By collecting and focusing the fluorescent light emitted by the probe of 143, the well 151 is functioning in a manner analogous to the lens 136 shown in FIG. 12. If desired, the conditioning optics 166 also include a lens or lenses that collect and focus light onto the detector 138 similar to the function performed by the lens (or lenses) 136 shown in FIG. 11.

Referring now to FIG. 13 another slide 170 is shown representing yet another embodiment of a biological slide for use in the analyzer 20. In this embodiment, the slide 170 may be constructed of a material that totally absorbs light having a frequency corresponding to the illumination light and is totally reflective of light having a frequency corresponding to the fluorescent light of a probe 174. Except for the different material of the slide 170, the embodiments of FIG. 12 and FIG. 13 are substantially the same. In this embodiment, the probe 174 is illuminated by light rays that enter the well 172 and directly strike the probes 174. Such light rays are illustrated by light rays 180 and 184 that are produced by scanning lasers 179 and 177, respectively. Light that strikes the side of the well 172 is totally absorbed, which is illustrated by light ray 186 in FIG. 13. As before, fluorescent light emanating from the probe 174 may travel directly toward and strike the detector 176, as illustrated by light rays 190, 192. Also, fluorescent light rays emitted from the probe 174 may be reflected from the side of well 172 toward the detector 176 as illustrated by light ray 194. Conditioning optics 178 are also provided between the detector 176 and probes 174 and preferably includes filters and lenses.

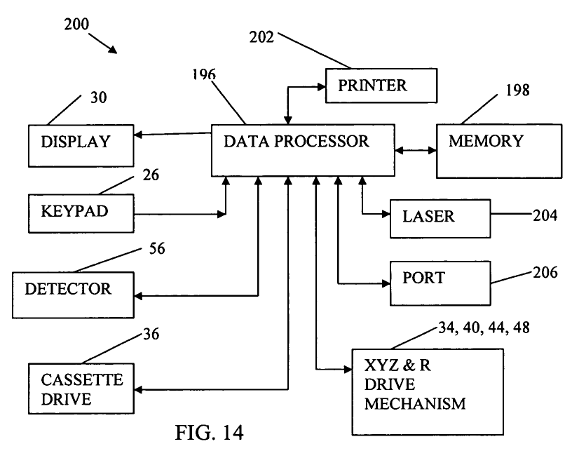

The internal operations of the analyzer 20 as described above may be understood at a more detailed level by reference to FIG. 14, a block diagram of the monitor and control system 200 that is used in the present invention. The system 200 includes a data processor 196 that receives, stores, and processes data and commands. Data is stored by the data processor 196 in a memory 198 and is accessed as necessary. Data and commands from a user are typically provided through the keypad 126 to the data processor 196, and information is transmitted from the data processor 196 to the user through the display 30. Data is transmitted from the detector 56 to the data processor 196. In the preferred embodiment, the detector 56 includes on board processing capabilities and therefore information is transmitted to the detector 56 as well.

The data processor 196 is also connected to monitor and control the cassette drive mechanism 36. When the user desires to insert a new slide into the analyzer 20, the command to open the cassette 24 is entered by the user through the keypad 26. The data processor 196 then issues commands to the cassette drive mechanism 36 causing it to extend the cassette 24 out of the housing 22. After a slide 28 has been inserted onto the cassette 24, the user inputs commands through the keypad 26, and in response to the user commands, the data processor 196 issues commands and controls the cassette drive mechanism 36 to translate the cassette 24 into the housing 22 and adjacent to the detector 56. In one embodiment, the slide 28 is read immediately when the slide 28 is placed on the cassette 24 and the cassette is withdrawn into the housing 22. In other embodiments, the data processor 196 will await commands from the user through keypad 26 before it begins of the process of reading the slides 28.

To begin the reading process, the data processor 196 issues commands to the X Y Z and R drive mechanisms 37, 40, 44, and 48. In response to these commands, the detector, such as detector 56 shown in FIG. 5, and the slide, such as the slide 28 shown in FIG. 6, are positioned adjacently. During a calibration process, the data processor 196 has been programmed to move the slide 28 to precisely place the probes 78 in alignment with the sensors 58. However, to double check the calibration and to account for possible mechanical misalignments, the data processor 196 queries the detector 56 and receives data corresponding to the light intensity detected at predetermined sensors that should be receiving a known fluorescence, such as sensors and 58-1,1 and sensor 58-5,5 shown in FIG. 5. As previously discussed, these two sensors are disposed over the upper left-most and lower right-most “A” probes 78 in the array 70. These two probes are emitting a known amount of fluorescent light. If the correct amount of light is not a detected by the sensors 58-1,1 and 58-5,5, the data processor 196 issues a series of search sequence commands causing the slide 28 to move in the X, Y, Z, R directions until the appropriate amount of light is received by the aforementioned of sensors. In this manner, the alignment of the slide 28 with the detector 56 is either verified or adjusted depending on the needs of each situation.

After the slide and detector have been aligned, the data processor 196 issues commands to turn on a source of light, such as a laser 204 shown in FIG. 14, and illuminate the slide 74. With a scanning pulsed laser, one may illuminate only the “A” probes 78 in the array 70 (FIG. 5), if desired, because they are the only probes that will be initially sensed. However, for reliability purposes, it is preferred to scan the array 70 (FIG. 5) and illuminate every probe 78. The data processor 196 then issues a command to extinguish the illumination source, such as laser 204, and immediately activate the detector 56 to detect fluorescent light that is emitted from the “A” probes 78 of the array 70. After the fluorescent light is measured by the detector 56, the data processor 196 issues commands causing the drive mechanisms 37, 44, 40 and 48 to reposition the slide so that the “B” probes 78 are aligned with the sensors 58. Then, the process of illuminating the probes 78, extinguishing the source of illumination, and reading the fluorescent light from the probes 78 is repeated as described above. Then, commands are issued by the data processor to move the slide 28 again and again to read the “C” and “D” probes 78 in the same manner. After all of the probes 78 in the array 70 have been read, the slide 28 is moved to read all of the arrays of probes 78 on the slide 28 such as arrays 72, 74 and 76.

After the entire slide has been read, the slide 28 may be removed from the analyzer 20 in response to commands issued by the user through the keypad 26, or the data processor 196 may be programmed to automatically eject the slide after it is read. The data that was obtained by the data processor 196 is initially stored in memory 198. However, under the control of the user 26, the data may be exported from the data processor 196 through such devices as a printer 202, “the display 30” or an electronic port 206 that communicates with other data processors and data collection devices. While specific examples of the invention have been discussed above, it will be understood that these examples are intended for the purpose of illustration only. It will be understood that the present intention is capable of numerous arrangements, modifications and substitutions of parts without departing from the scope and spirit of the invention as defined by the appended claims.

1. An analyzer for reading probes comprising:
a housing;
a slide carriage mounted for movement within the housing;
a slide disposed on the slide carriage;
a plurality of probes disposed on the slide in a probe pattern, each probe for generating a probe signal to indicate prior exposure to a predetermined substance or for not generating a probe signal to indicate the absence of prior exposure to the predetermined substance;
a detector for detecting probe signals from a plurality of probes located in a first pattern on the slide;
the probe pattern being denser than the first pattern and being configured in a shape corresponding to the probe pattern so that the detector can sense multiple subsets of the probes within a probe pattern on the slide; and
a drive mechanism for producing relative movement between the detector and the slide, the slide and detector moving relatively from a first position, in which the detector reads a first subset of probes within a probe pattern, to a second position in which the detector reads a second subset of probes within the probe pattern.

2. The analyzer of claim 1 further comprising a plurality of sensors arranged on the detector in the first pattern, each sensor being disposed for detecting a probe signal from a single probe when the plurality of sensors are aligned with a subset of the probes on the slide.

3. The analyzer of claim 1 further comprising a plurality of sensors arranged on the detector in the first pattern, the probe pattern containing a plurality of probes arranged on the slide in subsets of interlaced first patterns so that a single detector may be aligned sequentially with each subset of the probe pattern whereby all of the probes in the probe pattern may be read by aligning the plurality of sensors on the detector with each subset of the probe pattern.

4. The analyzer of claim 1 wherein the first pattern and the probe pattern comprise a plurality of locations are arranged in rows and columns.

5. The analyzer of claim 1 wherein the first pattern and the probe pattern comprise a plurality of locations arranged in a rectangular pattern in rows and columns.

6. The analyzer of claim 1 wherein the first pattern has of the shape of a pie section and the probe pattern is arranged on the slide in a radial orientation.

7. The analyzer of claim 1 wherein the slide is circular, the probe pattern is arranged on the slide in a radial orientation, and the first pattern on the detector is a subset of the probe pattern that may be repetitively aligned with the probe pattern by producing relative rotational movement between the slide and the detector.

8. The analyzer of claim 1 wherein the first pattern comprises a plurality of locations arranged in a truncated pie section, and wherein the probe pattern comprises a plurality of interlaced first patterns arranged radially about center point on the slide so that the slide may be read by placing and aligning the detector over one first pattern on the slide, reading the probes over which the detector is aligned, moving the slide and detector relatively to align the detector with subsequent sets of probes on the slide arranged in the first pattern.

9. The analyzer of claim 1 wherein the first pattern comprises a plurality of locations arranged in rows and columns wherein at least two different rows have different numbers of locations or wherein at least two different columns have a different numbers of locations so that the first pattern is irregular.

10. The analyzer of claim 1 wherein the first pattern comprises a plurality of locations arranged in rows and columns with a different number of locations in each row as compared to the other rows and a different number of locations in each column as compared to the other columns.

11. The analyzer of claim 1 wherein the first pattern comprises K number of locations arranged in X number of columns and Y number of rows, and the probe pattern comprises 4K number of locations arranged in 2X number of columns and 2Y number of rows, the drive mechanism being operable to move the slide relative to the detector so that all locations in the probe pattern are read after the slide is moved three times relative to the detector and each move is the distance from one location on the probe pattern to an adjacent location on the probe pattern.

12. The analyzer of claim 1 further comprising:
a source of electromagnetic radiation for illuminating the probes at selected times so that the probes are selectively illuminated or not illuminated;
the probes being constructed to not fluoresce to indicate prior exposure to selected materials and being constructed without any fluorescent material to indicate the absence of prior exposure to selected materials, so that certain probes emit a probe signal in the form of fluorescent light after being illuminated.

13. The analyzer of claim 1 further comprising alignment indicia disposed on the slide for being aligned with the detector to enable the detector to read selected probes.

14. The analyzer of claim 1 further comprising:
the probe pattern including a plurality of subsets, each subset being arranged in a first pattern;
a plurality of sensors arranged on the detector in the first pattern, each sensor being disposed for detecting a probe signal from a single probe when the plurality of sensors are aligned with a subset of the probes on the slide;
one or more indicia disposed on the slide in the location of one or more probes, the indicia producing a signal in the form of light for being detected by the sensors for aligning the detector such that the sensors are aligned over a subset of probes on the slide that are arranged in the first pattern.

15. The analyzer of claim 1 wherein the drive mechanism comprises an X drive mechanism, a Y drive mechanism and a Z drive mechanism for producing relative motion between the slide and detector in X, Y, and Z directions, respectively, where the X, Y, and Z directions are three non-parallel directions.

16. The analyzer of claim 1 wherein the drive mechanism comprise at least an R drive mechanism for producing relative rotational motion between the slide and the detector about an axis.

17. An analyzer for reading probes comprising:
a housing;
a slide carriage mounted for movement within the housing;
a slide disposed on the slide carriage;
a plurality of probes disposed on the slide in a probe pattern, each probe for generating a probe signal to indicate prior exposure to a predetermined substance or for not generating a probe signal to indicate the absence of prior exposure to the predetermined substance;
a detector for detecting probe signals from a plurality of probes located in a first pattern on the slide and generating detection of signals corresponding to the probe signals;
the probe pattern being denser than the first pattern and being configured in a shape corresponding to the first pattern so that the detector can sense multiple subsets of the probes within a probe pattern on the slide;
a drive mechanisms for moving the slide carriage and the slide from a first position, in which the detector reads a first subset of probes within a probe pattern, to a second position in which the detector reads a second a subset of probes within the probe pattern; and
a data processor connected to the drive mechanism and the detector for receiving the detection signals and for issuing command signals for controlling the drive mechanism based in part upon the detection signals.

18. The analyzer of claim 17 wherein said data processor is programmed:
to issue command signals causing the drive mechanism to move the detector to a first position,
to receive first detection signals from the detector in the first position and to issue command signals based upon the first detection signals causing the drive mechanism to move the detector to a second position.

19. The analyzer of claim 17 further comprising:
the probe pattern including a plurality of subsets, each subset being arranged in a first pattern;
a plurality of sensors arranged on the detector in the first pattern, each sensor being disposed for detecting a probe signal from a single probe when the plurality of sensors are aligned with a subset of the probes on the slide;
one or more indicia disposed on the slide in the location of one or more probes, the indicia producing a signal in the form of light for being detected by the sensors; and
the data processor being programmed to issue a first command causing the detector to move to a first position, to receive first detection signals corresponding to the light produced by the indicia, to issue a second command causing the detector to move to a second position based in part on the first detection signals.

20. The analyzer of claim 17 further comprising:
the probe pattern including a plurality of subsets, each subset being arranged in a first pattern;
a plurality of sensors arranged on the detector in the first pattern, each sensor being disposed for detecting a probe signal from a single probe when the plurality of sensors are aligned with a subset of the probes on the slide;
one or more indicia disposed on the slide in the location of one or more probes, the indicia producing a probe signal in the form of light for being detected by the sensors; and
the data processor being programmed to issue a first command causing the detector to move to a first position, to receive first detection signals corresponding to the light produced by the indicia, to issue a second command causing the detector to move to a second position based in part on the first detection signals, and to repetitively issue commands causing the detector to move to different positions based on received detection signals corresponding to the light produced by the indicia until the detection signals indicate that the light received from the first indicia falls within a predetermined intensity range.

21. The analyzer of claim 20 wherein the drive mechanisms is configured to move the slide carriage in X, Y, and Z, directions in response to commands issued by the data processor, where X, Y, and Z are three non-parallel directions.

22. The analyzer of claim 17 further comprising:
a keypad disposed on the housing and connected to the data processor for producing a key signals in response to input from a user, whereby a user may input commands through the keypad; but
the data processor being responsive to the key signals to collect data based upon the detection signals; and
a display disposed on the housing and connected to the data processor for displaying information to a user, said information corresponding to the commands provided by the user through the keypad and including data corresponding to the detection signals.

23. The analyzer of claim 17 further comprising output means for outputting data corresponding to the detection signals.

24. The analyzer of claim 17 further comprising a scanning laser disposed within the housing for scanning a laser beam over probe locations on the slide when the slide is positioned adjacent the detector, said scanning laser being operable to scan each of the probe locations in a first pattern that are being detected by the detector.

25. An analyzer for reading biological probes comprising:
a housing having a front wall and an opening through the front wall;
a cassette mounted within the housing for extending at least partially out of the housing through the opening in the front wall and for retracting at least partially into the housing through the opening;
cassette drive mechanism for moving of the cassette to the first and second positions of the cassette;
a user input for generating user commands in response to a user;
a display for displaying information to a user;
a slide carriage mounted on the cassette for movement with respect to the cassette;
a slide disposed on the slide carriage;
a plurality of probes disposed on the slide in a probe pattern, each probe for generating a probe signal to indicate prior exposure to a predetermined substance or for not generating a probe signal to indicate the absence of prior exposure to the predetermined substance;
a detector for detecting probe signals from a plurality of probes located in a first pattern on the slide and producing detection of signals corresponding to the probe signals;
a plurality of sensors disposed on the detector in the first pattern;
the probe pattern being more dense than the first pattern and being configured in a shape corresponding to a plurality of first patterns so that the detector can sense multiple subsets of the probes within a probe pattern on the slide; and
carriage drive mechanism for moving the slide carriage and the slide from a first position, in which the detector reads a first subset of probes within a probe pattern, to a second position in which the detector reads a second subset of probes within the probe pattern;
a data processor connected to the display, user input, detector, cassette drive mechanism, carriage drive mechanism, and an information output, said data processor for receiving a user commands from the user input, for transmitting information to the display and displaying information to the user on the display, for transmitting information causing the cassette drive mechanism to move the cassette between the first and second positions of the cassette, for transmitting commands to the carriage drive mechanism causing movement of the slide relative to the detector, for receiving detection signals and for sending information corresponding to the detection signals through the information output.

26. The analyzer of claim 25 further comprising;
indicia disposed on the slide in a location of a probe for producing alignment signals in the form of light that are detected by the detector, the detector producing alignment signals as part of the detection signals;
the data processor for receiving the alignment signals as part of the detection signals, for issuing a commands to at least the carriage drive mechanism causing the slide to move and aligning the sensors on the detector with the probes on the slide based upon the alignment signals.
